(19) United States
(12) Plant Patent Application Publication
(10) Pub. No.: US 2004/0049824 P1
(43) Pub. Date: Mar. 11, 2004

(54) Miniature rose plant named 'BRIfriends'

(76) Inventor: Dennis Bridges, Lawndale, NC (US)

Correspondence Address: Dennis Bridges, 2734 Toney Rd, Lawndale, NC, 28090, US

(21) Appl. No.: 10/237,287

(22) Filed: Sep. 9, 2002

(51) Int. Cl. A01H 5/00

(52) U.S. Cl. PLT/116

(57) ABSTRACT

A new and distinct variety of miniature rose plant characterized by a profusion of orange and yellow blend blooms on a vigorous compact plant with a continuous blooming habit.

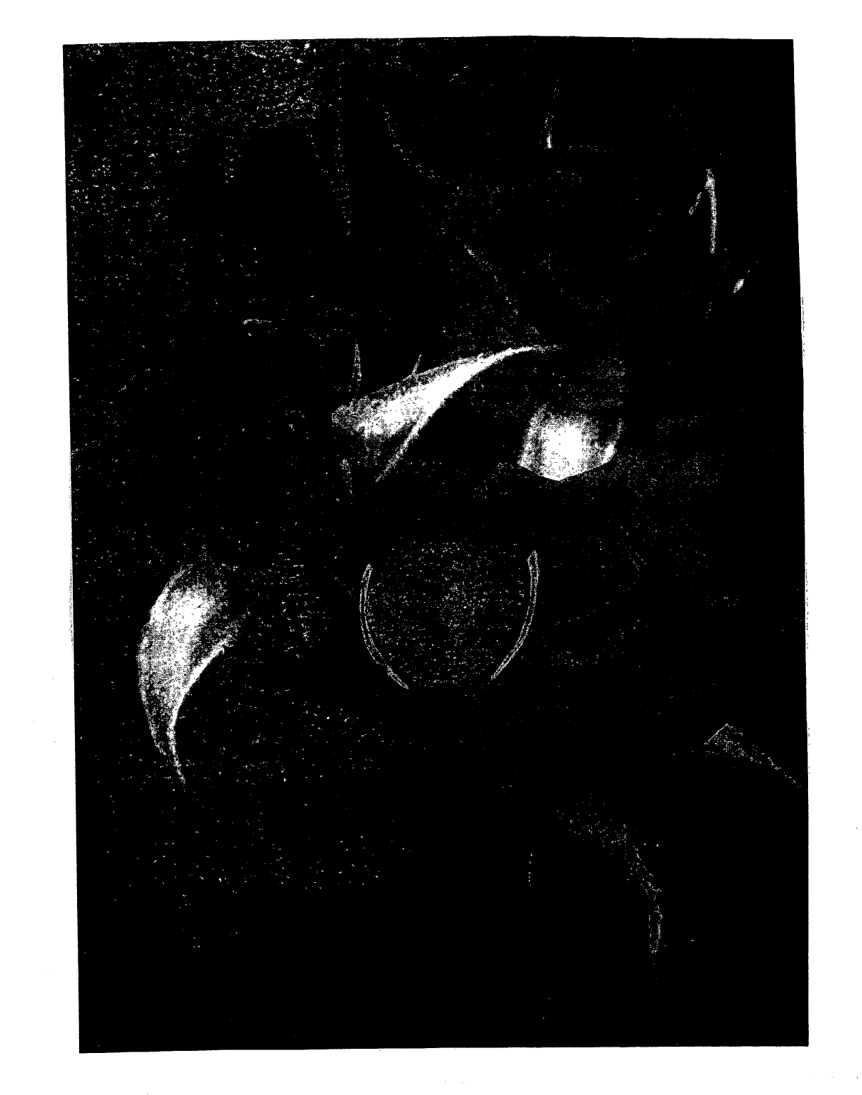

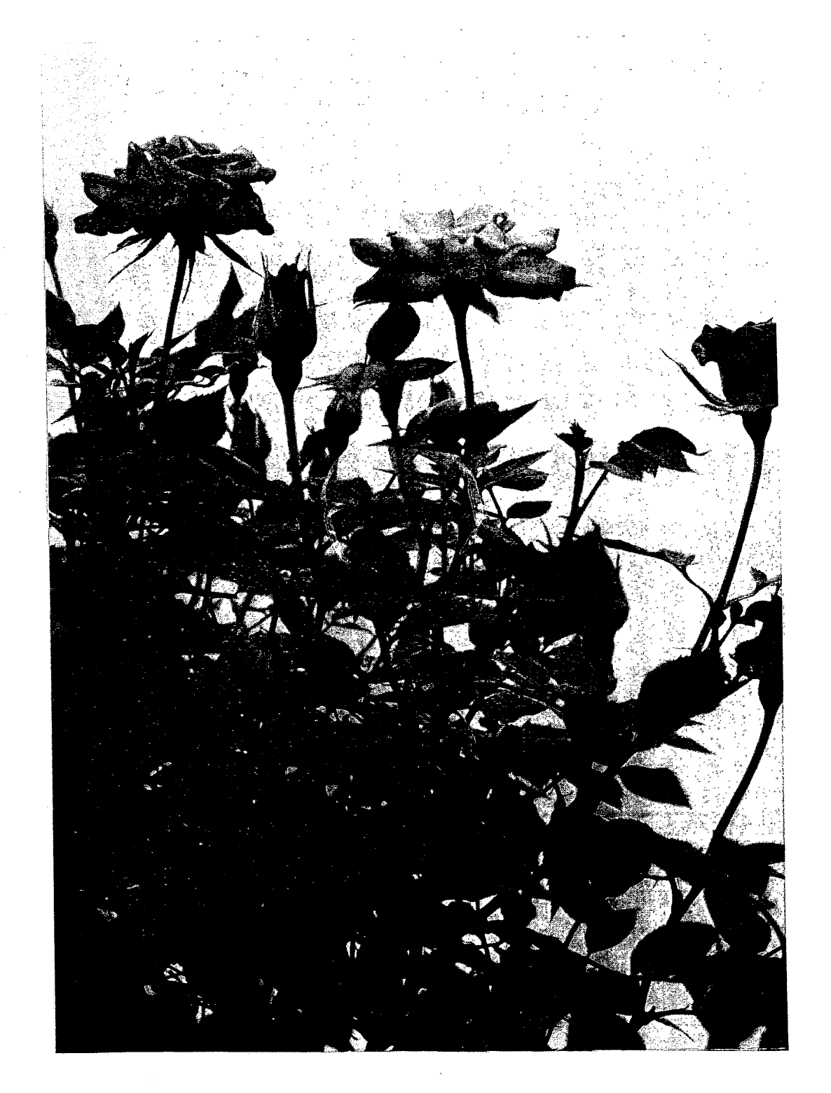

### BACKGROUND OF THE INVENTION

1. Field of the Invention

The present invention relates to a new and distinct variety of the miniature rose plant which has been given the international variety recognition designation “BRIfriends” and is to be marketed in this country under the synonym “Best Friends”

2. Plants Related to the Invention

The plant of this invention was originated as a result of a planned breeding program which had as a seed parent “Hot Tamale’” (U.S. Plant Pat. No. 9015) also of the miniature rose class. While the pollen patent of “BRIfriends” is not know with certitude, it was one of a number of selected miniature varieties from which pollen had been taken for crossing with “Hot Tamale’”, in the hope of attaining a new plant having some of the better traits of the seed parent and any one of the pollen parent plants involved in the program. The plant of this disclosure is a selection from the progeny of the stated cross(es), and has been asexually reproduced by the taking and rooting of stem cuttings at Lawndale, N.C., as performed characteristics and distinctions come true to form and are established and transmitted through succeeding propagation's establishing ‘BRIfriends’ is stable.

### SUMMARY OF THE INVENTION

Among the new and novel characteristics which are possessed by this plant, singly and in combination, which distinguish it from its parents and all other plants of which I am aware are:

1. Blooms of deep orange and yellow, that are well formed and blemish resistant. Showing exceptional form and substance.

2. Abundant blooms borne singly and in clusters of up to 5 blooms, on a compact plant that blooms continuously.

3. An abundance of glossy, medium to dark green foliage which pleasingly contrasts with and highlights the orange and yellow blend blooms.

4. Higher than usual disease resistance to common rose diseases with a higher than usual level of vigor, and growing to a height which is typical of roses of this market class to produce a plant which attractively appoints the home garden while providing a plant that is well suited for the garden and container gardening.

### BRIEF DESCRIPTION OF THE DRAWING

The two sheets of the accompanying drawing are photographs, one shows typical specimens of the flowers of the new variety in different stages of development as well as the coloration, the second shows typical specimens of the flowers on the plant in different stages of development as well as the coloration thereof as accurately as possible in color illustrations of this type.

### BOTANICAL DESCRIPTION OF THE DRAWING

The following is a detailed botanical description of my new variety of miniature rose. With color terminology in accordance with The Royal Horticultural Society Colour Chart (R.H.S.C.C.), except where common dictionary terms of color definition are employed.

Type: Hardy, dwarf, compact, bushy, seedling, greenhouse, outdoors, cut flower, garden decoration, potted plant.

Commercial class: Miniature Rose.

International recognition name: “BRIfriends”.

Commercial synonym: “Best Friends”.

Breeding.—Seedling.

Seed parent.—“Hot Tamale”.

Pollen parent.—Unknown (select pollen).

### FLOWER

Observations were made from specimens grown outdoors at Cleveland County, North Carolina, USA, in June 2002.

Type: Miniature Rose.

Blooming habit: Continuous.

Bud:

Size.—Medium.

Form.—Urn shaped.

Color.—When sepals first divide: Near to 42A with near to 20A When petals begin to unfurl: Near to 42A on petal edges, near to 20A at base of petals When half-blown: inside of petals: Near to 46C blending near to 20A reverse side of petals: Petals near to 46C blending near to 20A at base of petals.

Sepals.—Characteristics, appearance, length in relation to bud: As long as bud.

Peduncle.—Length ⅝ inch Aspect: Strong Strength: Erect Color: Near to 143A.

Bloom:

Size:

Average when fully expanded.—1⅛ inches.

Borne.—Singly and in sprays of 3 to 5 blooms.

Form.—High centered, opening with petals reflexing, becoming flat with age.

Petalage.—20 to 23.

Color.—(during first two days) Inner petals at center of flower: Near to 45C Outer petals: Near to 42A on petal edges, blending near to 46C Base of petals: Near to 20A General tonality from a distance: Orange blend, near to 43A and near to 20A.

Color change.—As bloom ages: Lightens slightly, near to 46D) and near to 20A General tonality: Orange blend, near to 46C and 46D blending with near to 21C After three or more days: Lightens more, near to 47B, blending with more yellow near to 21C.

Petals:

Texture.—Thick.

Appearance.—Velvety inside — satiny outside.

Form.—Oval, pointing on edge.

Arrangement.—Regular.

Petaloids in center.—Few.

Persistence.—Drop off cleanly after extensive time on bush (4 to 6 days).

Fragrance.—Slight.

Lasting quality.—Blooms last 4 to 6 days on plant and 7 to 9 days as a cut flower.

### REPRODUCTIVE ORGANS

Stamens, filaments and anthers.

Arrangement.—Regularly arranged around styles.

Color.—Yellow.

Pollen: Color: Yellow.

Styles: Uneven length, thin, bunches.

Stigmas: Yellow.

Hips: None.

Shape.—

Size.—

Color when mature.—

Do seeds protude.—

### PLANT

Form: Upright.

Growth:

Height attained.—20 to 24 inches tall.

Foliage:

Size.—Medium.

Quantity.—Abundant.

Color.—New foliage: upper side: Near to 147B edged with near to 180A under side: Near to 147B with more near to 180A Old foliage: upper side: Near to 136B under side: Near to 136B.

Shape: Oval, pointed.

Texture: Upper side: Smooth Under side: Smooth, veins raised slightly.

Edge: Serrated.

Serration: Regular.

Leaf stem: Color: Near to 137C Under side: Near to 137C.

Stipules: ½ inch long with small points.

Resistance to disease: Blackspot: Good.

Mildew.—Good.

Rust.—Good.

Wood:

New wood.—Color: Near to 144A Bark: Smooth.

Old wood.—Color: Near to 137B Bark: Smooth.

Thorns:

Quantity.—on main stalks from base: Few On laterals from stalk: More on lateral stalks Form: Flat at base, sharp, pointing slightly downward Length: ½ inch Color (when young): Near to 146C Position: Irregular.

Prickles.—on main stalks: No moss on canes on laterals: No moss on canes on peduncle: Many small prickles.

Winter hardiness: Winter hardy with little or no die back. Lows — average winter temperature teens to an occasional minus temperature.

I claim:

1. A new and distinct variety of rose plant of the miniature rose class, substantially as herein shown and described, characterized particularly by an abundance of deep orange and yellow blend blooms, unusually strong substance, firm thick petals, borne singly and in sprays on a compact plant that blooms continuously, with semi-glossy dark green foliage, said plant being easy to propagate from cuttings, and said blooms being long lasting both on the plant and as cut flowers.
